(19) United States
(12) Patent Application Publication
(10) Pub. No.: US 2026/0053561 A1
(43) Pub. Date: Feb. 26, 2026

(54) MICROWAVE ABLATION DEVICE WITH OPTICAL SENSOR

(71) Applicant: Neuwave Medical, Inc., Madison, WI (US)

(72) Inventors: Emily FORAN, Sun Prairie, WI (US); Todd KIEFER, Madison, WI (US); Scott JOHNSON, Madison, WI (US); Matthew THIEL, Verona, WI (US); Robert KNOWLTON, Madison, WI (US)

(73) Assignee: Neuwave Medical, Inc., Madison, WI (US)

(21) Appl. No.: 18/811,101

(22) Filed: Aug. 21, 2024

(51) Int. Cl. A61B 18/18; A61B 18/00; A61B 34/20

(52) U.S. Cl. CPC A61B 18/1815; A61B 2018/00577; A61B 2018/00797; A61B 2018/00821; A61B 2018/00898; A61B 2034/2061

(57) ABSTRACT

A system is disclosed including an energy delivery device and a controller. The energy delivery device includes a cannula, an antenna extending from the cannula and operable to deliver energy to tissue of the patient, and an optical sensor including Fiber Bragg Gratings (FBGs). The cannula and the antenna are insertable through skin of a patient to an insertion depth. The controller is in communication with the optical sensor and operable to receive temperature measurements from the optical sensor and determine the insertion depth based on the received temperature measurements.

### BACKGROUND

Ablation is an important therapeutic strategy for treating certain tissues, such as benign and malignant tumors, cardiac arrhythmias, cardiac dysrhythmias, and tachycardia. Some ablation systems utilize radio frequency (RF) energy as the ablating energy source. However, RF energy has several limitations, including the rapid dissipation of energy in surface tissues resulting in shallow “burns” and failure to access deeper tumor or arrhythmic tissues. Another limitation of RF ablation systems is the tendency of eschar and clot formation on the energy emitting electrodes, which limits further deposition of electrical energy.

More recently, microwave energy is being used as the ablating energy source in ablation systems. Microwave energy is an effective energy source for heating biological tissues, and is used in applications such as cancer treatment and preheating of blood prior to infusions. One advantage of microwave energy over RF is the deeper penetration into tissue, insensitivity to charring, lack of necessity for grounding, more reliable energy deposition, faster tissue heating, and the capability to produce much larger thermal lesions than RF, which greatly simplifies the actual ablation procedures.

Current microwave ablation devices typically include multiple thermocouples, each of which terminates at a discrete location along the device for measuring local temperatures thereof. While these thermocouples do provide some amount of temperature data, they do not provide a complete picture regarding the temperature profile along the device. Due to the limited number of temperature sensing points, a user may not properly understand the temperature gradient along the device due to the emitted microwave energy. This lack of understanding may cause the emitted microwave energy to burn the skin at the device entry point, for example, which would be detrimental to the patient.

Accordingly, there is a need for improved systems and devices for measuring temperature along a microwave ablation device.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an energy delivery system that may be used in accordance with the principles of the present disclosure.

FIG. 2 is a schematic diagram of an ablation probe of the energy delivery system of FIG. 1 including an optical sensor, according to at least one aspect of the present disclosure.

FIG. 3 is a partial, detailed view of the ablation probe of FIG. 2, according to at least one aspect of the present disclosure.

FIG. 4 is the ablation probe of FIG. 2 inserted through the skin of a patient at a first time, according to at least one aspect of the present disclosure.

FIG. 4A is a graph of temperature measurements measured by the ablation probe at the first time, according to at least one aspect of the present disclosure.

FIG. 5 is the ablation probe of FIG. 2 inserted through the skin of the patient at a second time later than the first time, according to at least one aspect of the present disclosure.

FIG. 5A is a graph of temperature measurements measured by the ablation probe at the second time, according to at least one aspect of the present disclosure.

FIG. 6 is the ablation probe of FIG. 2 inserted through the skin of the patient at a third time later than the second time, according to at least one aspect of the present disclosure.

FIG. 6A is a graph of temperature measurements measured by the ablation probe at the third time, according to at least one aspect of the present disclosure.

FIG. 7 is the ablation probe of FIG. 2 inserted through the skin of the patient at a fourth time later than the first time, according to at least one aspect of the present disclosure.

FIG. 7A is a graph of temperature measurements measured by the ablation probe at the fourth time, according to at least one aspect of the present disclosure.

### DETAILED DESCRIPTION

The present disclosure is related to systems and methods for delivering energy to tissue for ablation operation and, more particularly, to systems and methods for controlling the temperature of the skin to prevent burns thereto during an energy delivery procedure.

The present disclosure is related to comprehensive systems, devices, and methods for delivering energy (e.g., microwave energy, radiofrequency energy, laser, focused ultrasound, plasma, etc.) to tissue for a wide variety of applications including medical procedures (e.g., percutaneous or surgical). Example medical procedures that may benefit from the embodiments described herein include, but are not limited to, tissue ablation, resection, cautery, vascular thrombosis, intraluminal ablation of a hollow viscus, cardiac ablation for treatment of arrhythmias, electrosurgery, tissue harvest, cosmetic surgery, intraocular use, or any combination thereof.

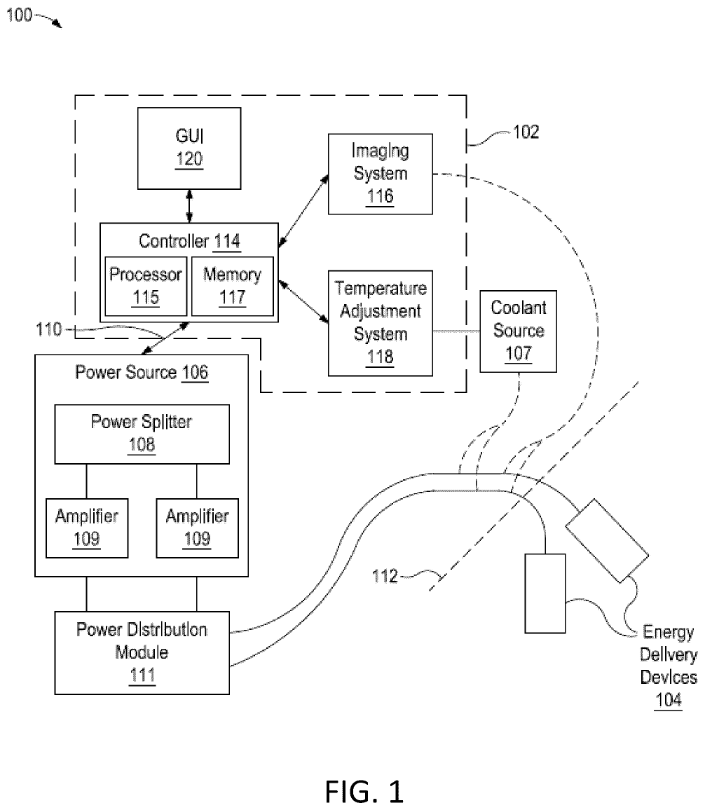

FIG. 1 is a block diagram of an example energy delivery system 100 that may incorporate the principles of the present disclosure. As illustrated, the energy delivery system 100 (hereafter “the system 100”) includes a control system 102 and one or more energy delivery devices or “ablation probes” 104 (two shown) designed to deliver (emit) energy to a target tissue region of a patient. The system 100 further includes a power source or generator 106 communicably coupled to the control system 102 and the ablation probes 104 to direct, control, and deliver (provide) electrical power thereto. In some applications, the power source 106 may include a power splitter 108 that receives power from an external power source (e.g. a wall outlet) and directs power to one or more amplifiers 109, which amplify the voltage, current, or power from the power splitter 108. While two amplifiers 109 are shown, the power source 106 may include less than two amplifiers (e.g. one amplifier) or more than two amplifiers (e.g. three, four, or five amplifiers, for example). Each amplifier 109 may be coupled to a corresponding ablation probe 104 via a power distribution module 111, which provides strain relief to the cabling extending from the amplifiers 109 to the ablation probes 104. The power distribution module 111 may be coupled to a structure in the operating room, such as a surgical bed, and may house connection hardware of the probes 104.

The components of the system 100 are connected via one or more cables or transmission lines 110. Moreover, the ablation probes 104 are designed to operate within a sterile field facilitated by the use of a sterile field barrier 112 that separates the ablation probes 104 from the remaining components of the system 100. The sterile field barrier 112 creates the sterile field, which includes any region permitting access only to sterilized items (e.g., sterilized devices, sterilized accessory agents, sterilized body parts, etc.). The sterile field barrier 112 hinders entry of non-sterile items into the sterile field, and the ablation probes 104 are configured for operation within the sterile field.

The control system 102 is configured to monitor, control, and provide feedback concerning operation of the system 100. As illustrated, the control system 102 includes at least a controller 114, an imaging system 116, and a temperature adjustment system 118. The control system 102 may further include a graphical user interface (GUI) 120, such as a touchscreen interface, which can be accessed by a user (e.g., a surgeon, a nurse, bedside assist, etc.) to operate the system 100. In some applications, the control system 102 may be mounted to or otherwise form part of a portable cart or “procedure cart,” and the GUI 120 may be arranged in a display region for operating and/or monitoring the components of the system 100.

The controller 114 may include a processor 115 and a memory or memory device 117 comprising any storage media readable by the processor 115. The memory 117 may store software or software instructions executable by the processor 115 to carry out functions and operations of the system 100. Examples of the memory 117 include, but are not limited to, random access memory (RAM), read-only memory (ROM), computer chips, optical discs (e.g., compact discs (CDs), digital video discs (DVDs), etc.), magnetic disks (e.g., hard disk drives (HDDs), floppy disks, ZIP® disks, etc.), magnetic tape, and solid state storage devices (e.g., memory cards, “flash” media, etc.). As used herein, the term “computer readable medium” refers to any device or system for storing and providing information (e.g., data and instructions) to the processor 115. Examples of computer readable media include, but are not limited to, optical discs, magnetic disks, magnetic tape, solid-state media, and servers for streaming media over networks.

Based on instructions provided by the software, the controller 114 may be configured to regulate the amount of energy (e.g., microwave energy) provided to a tissue region by the ablation probes 104 by monitoring characteristics of the tissue region, such as the size and shape of a target tissue, the temperature of the tissue region, etc. The controller 114 interacts with the ablation probes 104 to raise or lower (e.g., tune) the amount of energy delivered to the tissue region. The controller 114 may also be configured to prime coolants for distribution into the ablation probes 104 such that the coolant is delivered at a desired temperature.

In some applications, the type of tissue being treated is inputted into the software for purposes of allowing the controller 114 to regulate (e.g., tune) the delivery of microwave energy to the tissue region based upon pre-calibrated methods for that particular type of tissue or tissue region. In other embodiments, however, the type of probe selected for the particular procedure may be specifically tuned to a specific tissue type, and projected ablation sizes may be based on tissue type. In such embodiments, the controller 114 may not control power delivery based on tissue type. In yet other embodiments, the controller 114 generates a chart or diagram based upon a particular type of tissue or tissue region displaying characteristics useful to a user of the system.

The controller 114 may allow a user to choose power, duration of treatment, different treatment algorithms for different tissue types, simultaneous application of power to multiple probes 104, coherent and incoherent phasing, etc. The controller 114 may also be configured to create a database of information (e.g., required energy levels, duration of treatment for a tissue region based on particular patient characteristics, etc.) pertaining to ablation treatments for a particular tissue region based upon previous treatments with similar or dissimilar patient characteristics.

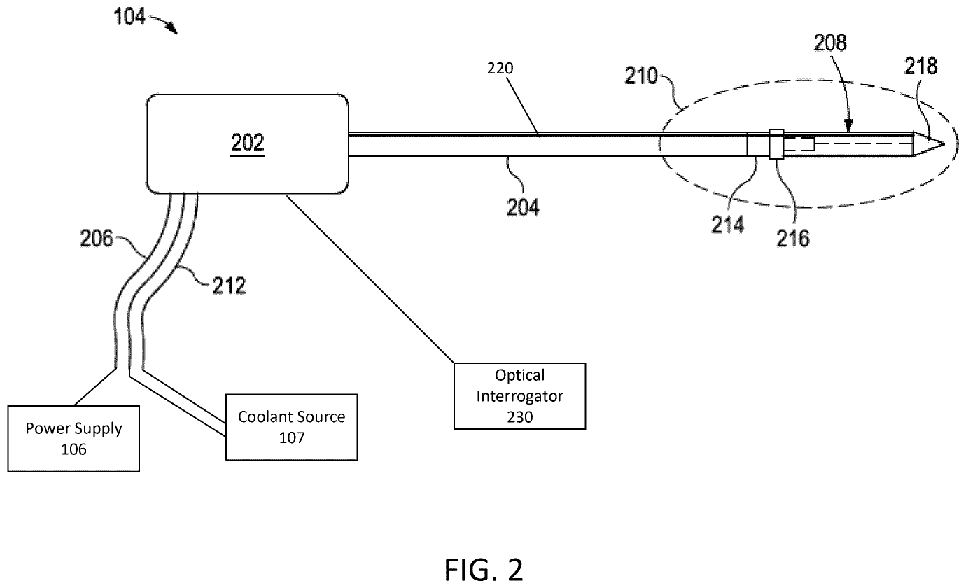

FIG. 2 is a schematic diagram of an ablation probe 104 that may be used in accordance with the principles of the present disclosure, according to one or more embodiments. As indicated above, the ablation probe 104 may be configured to deliver (emit) energy (e.g., microwave energy, radiofrequency energy, radiation energy) to a target tissue region. As illustrated, the ablation probe 104 includes a handle housing 202 and an elongate shaft or probe cannula 204 extending distally from the handle 202. In some embodiments, the probe cannula 204 may be rigid (for percutaneous use, for example) or flexible (for transport through a bronchoscope, for example). In some embodiments, the probe 104 may not include the handle housing 202.

A cable or cable assembly 206 may be operatively coupled to the handle 202 and configured to convey electrical power thereto. The cable assembly 206 may extend from the power source 106 (FIG. 1), for example, and may provide the power sufficient to operate the ablation probe 104. An antenna 208 is provided at the distal end of the probe cannula 204 and receives electrical power from the cable assembly 206 to emit energy (e.g., microwave energy) to a target tissue region and thereby generate an ablation zone 210. The material of the antenna 208 is durable and provides a high dielectric constant. In some applications, the material of the antenna 208 is zirconia and/or a functional equivalent of zirconia. In at least one application, the ablation probe 104 includes two or more separate antennae 208 attached to the same or different power supplies.

In some applications, a cooling tube 212 is operatively coupled and configured to convey a cooling fluid or “coolant” to the handle 202. The handle 202 may be configured to control conveyance of the cooling fluid into and out of the probe cannula 204 to help regulate a temperature of the antenna 208 and the ablation zone 210. Example cooling fluids include, but are not limited to, water, glycol, air, inert gases (e.g., helium), carbon dioxide, nitrogen, sulfur hexafluoride, ionic solutions (e.g., sodium chloride with or without potassium and other ions), dextrose in water, Ringer's lactate, organic chemical solutions (e.g., ethylene glycol, diethylene glycol, or propylene glycol), oils (e.g., mineral oils, silicone oils, fluorocarbon oils), liquid metals, freons, halomethanes, liquified propane, other haloalkanes, anhydrous ammonia, sulfur dioxide, or any combination thereof.

In some applications, the ablation probe 104 may include a stick region 214, alternately referred to as a “tissue-loc” region, provided on the probe cannula at, near, or proximal to the antenna 208. The stick region 214 is designed to attain and maintain a temperature that accommodates adherence of a tissue region onto its surface. More specifically, the stick region 214 may operate as an anchoring element having a circulating agent or “coolant” (e.g., a gas delivered at or near its critical point; CO₂) that freezes the interface between the stick region 214 and the adjacent tissue, thereby sticking (maintaining, locking, etc.) the antenna 208 in place during operation. The coolant may be provided to the stick region 214 via the cooling tube 212 from the coolant source 107. Once a pre-determined low temperature is reached at the stick region 214, contact with adjacent tissue causes the tissue to adhere to the stick region 214, thereby resulting in attachment of the energy delivery device 104 to the tissue. During ablation, as the tissue warms, the antenna 208 remains secured to the tissue region due to tissue desiccation and charring. The stick region 214 may be made of any material able to attain and maintain a temperature such that upon contact with tissue induces adherence of the tissue onto the stick region 214. Example materials for the stick region 214 include, but are not limited to, a metal.

In some applications, the ablation probe 104 may further include a plug region 216 provided on the probe cannula 204 at or near the antenna 208. In at least one application, as depicted, the plug region 216 may be provided distal to the stick region 214 and otherwise interposing the stick region 214 and the antenna 208. The plug region 216 may be configured to prevent a reduction in temperature resulting from the cooled probe cannula 204 and the stick region 214 from affecting (e.g., reducing) the temperature within the antenna 208. Accordingly, the plug region 216 separates interior portions of the ablation probe 104 to prevent cooling or heating of a portion or portions of the device 104 while permitting cooling or heating of other portions. The plug region 216 may be made of an insulative material capable of being in contact with a material or region having a low temperature without having its temperature significantly reduced. Example insulative materials for the plug region 216 include, but are not limited to, a synthetic polymer (e.g., polystyrene, polyicynene, polyurethane, polyisocyanurate), aerogel, fiberglass, cork, or any combination thereof.

In some applications, the ablation probe 104 may further include a sharp stylet tip or “stylet” 218 positioned at the distal end of the antenna 208 and otherwise forming the distal end of the ablation probe 104. When included, the stylet 218 is designed to facilitate percutaneous insertion of the ablation probe 104. The stylet 218 may be made of a variety of rigid or hardened materials including, but not limited to, a hardened resin, a metal (e.g., titanium or an equivalent of titanium, stainless steel, etc.), a ceramic, or any combination thereof. In at least one application, the stylet 218 may be braised to zirconia or an equivalent of zirconia. In such applications, the stylet 218 may comprise an extension of a metal portion of the antenna 208 and may be electrically active.

In some applications, the ablation probe 104 may have a coaxial transmission line positioned within the antenna 208, and a coaxial transmission line connecting with the antenna 208. In other embodiments, the ablation probe 104 may comprise a triaxial microwave probe with optimized tuning capabilities. The ablation probe 104 may be the same as or similar to any of the energy delivery devices described in U.S. Pat. No. 11,638,607, entitled “ENERGY DELIVERY SYSTEMS AND USES THEREOF”, which issued on May 2, 2023, the contents of which are hereby incorporated by reference in their entirety herein.

Referring again to FIG. 1, the control system 102 further includes the imaging system 116, which is in communication with the controller 114 and comprises one or more imaging devices. Example imaging devices include, but are not limited to, endoscopic devices, stereotactic computer assisted neurosurgical navigation devices, thermal sensor positioning systems, motion rate sensors, steering wire systems, intraprocedural ultrasound, interstitial ultrasound, microwave imaging, acoustic tomography, dual energy imaging, fluoroscopy, computerized tomography magnetic resonance imaging, nuclear medicine imaging devices triangulation imaging, thermoacoustic imaging, infrared and/or laser imaging, or electromagnetic imaging. In some embodiments, the system 100 uses endoscopic cameras, imaging components, and/or navigation systems that permit or assist in placement, positioning, and/or monitoring of the ablation probes 104.

In some applications, the imaging system 116 may be configured to monitor ablation procedures, such as monitoring the amount of ablation occurring within a particular tissue region(s) undergoing a thermal ablation procedure. The monitoring includes, but is not limited to, MRI imaging, CT imaging, ultrasound imaging, nuclear medicine imaging, and fluoroscopy imaging. The software may be designed to automatically obtain images of a tissue region (e.g., MRI imaging, CT imaging, ultrasound imaging, nuclear medicine imaging, fluoroscopy imaging), automatically detect any changes in the tissue region (e.g., blood perfusion, temperature, amount of necrotic tissue, etc.), and based on the detection to automatically adjust the amount of energy delivered to the tissue region through the ablation probes 104.

The power source 106 may be configured to supply the energy required to operate the system 100. The power source 106 is also configured to supply energy to the ablation probes 104, such as microwave energy, radiofrequency energy, radiation, cryo energy, electroporation, high intensity focused ultrasound, or any combination thereof. In accordance with principles of the present disclosure, the power source 106 supplies microwave energy to the ablation probes 104 for purposes of tissue ablation. More specifically, power may be supplied to the ablation probes 104, but the microwave energy is generated in a microwave generator and sent to the antenna 208. In some applications, the power source 106 may include one or more energy generators configured to provide as much as 140-150 watts of microwave power of a frequency of from 915 MHz to 5.8 GHz, although the present invention is not so limited. The power splitter 108 may comprise a power distribution system operable to distribute the energy from the power source 106 to the ablation probes 104. The power splitter 108 may be configured to provide varying energy levels to different regions of the ablation probes 104.

The temperature adjustment system 118 may be configured to use coolant systems and cooling fluids to help reduce undesired heating within and along the ablation probes 104. In particular, the temperature adjustment system 118 may include the cooling tube 212 (FIG. 2) and may communicate with the handle 202 (FIG. 2) of each probe 104 to control conveyance of the cooling fluid into and out of the probe cannula 204 (FIG. 2), and thereby help regulate a temperature of the antenna 208 (FIG. 2) and the ablation zone 210 (FIG. 2). In some applications, the temperature adjustment system 118 may also be configured to communicate with the handle 202 to operate the stick region 214 and thereby attain and maintain a temperature that accommodates adherence of tissue onto its surface.

The temperature adjustment system 118 may be configured to use coolant systems (like the coolant source 107) and cooling fluids to help reduce undesired heating within and along the ablation probes 104. In particular, the temperature adjustment system 118 may include the coolant source 107 (FIG. 1) and the cooling tube 212 (FIG. 2) and may communicate with the handle 202 (FIG. 2) of each probe 104 to control conveyance of the cooling fluid into and out of the probe cannula 204 (FIG. 2), and thereby help regulate a temperature of the antenna 208 (FIG. 2) and the ablation zone 210 (FIG. 2). In some applications, the temperature adjustment system 118 may also be configured to communicate with the handle 202 to operate the stick region 214 and thereby attain and maintain a temperature that accommodates adherence of tissue onto its surface. For instance, in some embodiments, a user provides an input to an input interface, such as the GUI 120. Based on the user input, the temperature adjustment system 118 can control the coolant source 107 to provide coolant to the stick region 214, thereby adhering the stick region 214 to the adjacent tissue.

In some embodiments, the temperature adjustment system 118 can monitor (continuously or intermittently) the real-time temperature of the ablation probes 104. In such embodiments, the temperature adjustment system 118 may communicate with a temperature sensor 220, discussed in more detail below, to monitor the temperature at several points along the antenna 208 to estimate ablation status, cooling status, or safety checks. In some applications, monitoring the temperature at several points along the antenna 208 may help determine the geographical characteristics of the ablation zone 210 (FIG. 2), such as diameter, depth, length, density, width, etc., based upon the tissue type, and the amount of power used in the ablation probe 104. In other embodiments, or in addition thereto, the temperature may be measured not only at specific points along the probe cannula 204, but continuously along its entire length.

The temperature adjustment system 118 may also be configured to monitor the temperature of a tissue region (e.g., tissue being treated, surrounding tissue). This may prove advantageous in helping to determine the status of the procedure (e.g., the end of the procedure). The temperature adjustment system 118 may communicate with the controller 114 to provide real-time temperature information to a user and display such measurements on the GUI 120. In at least one embodiment, based on the temperature data obtained by the temperature adjustment system 118, the controller 114 may be configured to autonomously adjust operation of the system 100 appropriately.

As discussed above, current microwave ablation devices typically include multiple thermocouples, such as three, four, or five thermocouples, for example, each of which terminates at a discrete location along the device for measuring local temperatures thereof. While these thermocouples do provide some amount of temperature data, they do not provide a complete picture regarding the temperature profile along the device. Due to the limited number of temperature sensing points, a user may not properly understand the temperature gradient along the device due to the emitted microwave energy. This lack of understanding may cause the emitted microwave energy to burn the skin at the device entry point, for example, which would be detrimental to the patient.

Accordingly, the ablation probe 104 further comprises an optical sensor 220 for measuring a plurality of temperatures along a length of the ablation probe 104. In some embodiments, the optical sensor 220 extends from the handle housing 202, along the probe cannula 204, and terminates at a distal end of the antenna 208. In alternative embodiments, the optical sensor 220 terminates at the stylet 218, which can be advantageous, as discussed in more detail below. In some embodiments, the optical sensor 220 extends within (through) the cannula 204 and the antenna 208. In some such embodiments, the antenna 208 may include a conductor defining a channel therein and the optical sensor 220 extends through the channel. In some other embodiments, the optical sensor 220 is coupled to and extends along the exterior of the probe cannula 204 and the antenna 208 (and, in some embodiments, the stylet 218, as referenced above).

The control system 102 further comprises an optical interrogator 230 that can be electrically powered by the power source 106 and can be controlled by the controller 114. The optical interrogator 230 is operably coupled to the optical sensor 220 such that the optical interrogator 230 can provide (transmit) an optical (light) signal therethrough and receive a reflected light signal therefrom, as will be discussed in more detail below.

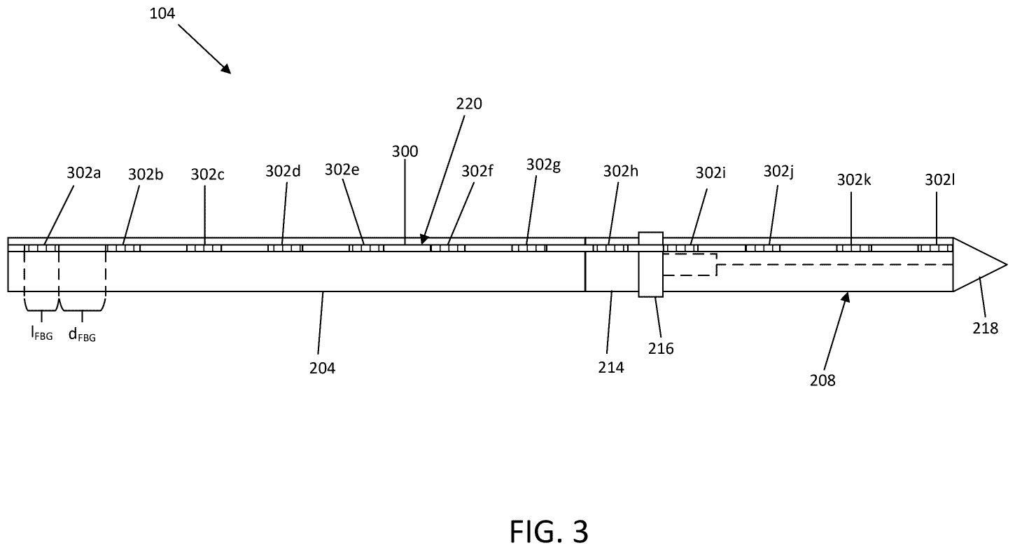

FIG. 3 is a partial, detailed view of the ablation probe of FIG. 2, according to at least one aspect of the present disclosure. In some embodiments, the optical sensor 220 includes an optical fiber 300 that includes a plurality of Fiber Bragg Grates (FBGs) or “temperature sensor portions” 302a-l defined along the optical fiber 300. An FBG is a type of reflector defined in a short segment of an optical fiber (such as the optical fiber 300) that reflects particular wavelengths of light and transmits others therethrough. Based on the wavelengths reflected, an optical interrogator (such as the optical interrogator 230) can determine various information associated with the FBG, such as the temperature at the FBG or the strain experienced by the FBG, as examples. Thus, light reflected back to the interrogator 230 from a particular FBG provides an indication of temperature or strain at that FBG based on the wavelength of the reflected light.

In some embodiments, each of the FBGs 302a-l are defined along a length l_{FBG} of the optical fiber 300 and are spaced apart a distance d_{FBD} from one another along the optical fiber 300. In some such embodiments, the FBGs 302a-l are defined along the same length l_{FBG} of the optical fiber 300 and are positioned the same distance d_{FBD} apart from one another along the optical fiber 300. In other such embodiments, the FBGs 302a-l are defined along variable lengths l_{FBG} of the optical fiber 300 and are positioned at variable distances d_{FBD} apart from one another along the optical fiber 300. In some embodiments, the FBGs 302a-l include a uniform grating, a chirped grating, a tilted grating, or a superstructure grating, or combinations thereof. In some embodiments, some of the FBGs are defined along the optical fiber 300 at points of interest of ablation probe 104, such as at the stick region 214 (FBG 302h), at a proximal end 304 of the antenna 208 (FBG 302i), and a distal end 306 of the antenna 208 (302l), as examples. In embodiments where the optical sensor 220 terminates at the stylet 218, as referenced above, an FBG may be defined at the stylet 218. Defining an FBG at the stylet 218 enables the controller 114 to monitor the amount of strain experienced by the stylet FBG, such as when the user attempts to percutaneously insert the ablation probe 104 into the patient, for example. While twelve FBGs 302a-l are shown and described, it should understood that the optical fiber 300 may have fewer than twelve FBGs (four, six, eight, or ten FBGs, for example) or more than twelve FBGs (fourteen, sixteen, or eighteen FBGs, for example). The use of more than one optical fiber, each with one or more FBGs, is also contemplated.

In operation, the optical interrogator 230 can transmit an optical (light) signal through the optical fiber 300 (or optical fibers if more than one is used). As the optical signal traverses the optical fiber 300, particular wavelengths of light are reflected at each of the FBGs 302a-l and transmitted back to the optical interrogator 230. Based on the detected reflected signals, the optical interrogator 230 can determine various information associated with each of the FBGs 302a-l, such as the temperature or strain at each of the FBGs. This detected information can be used by the controller 114 for a variety of purposes, as discussed in more detail below.

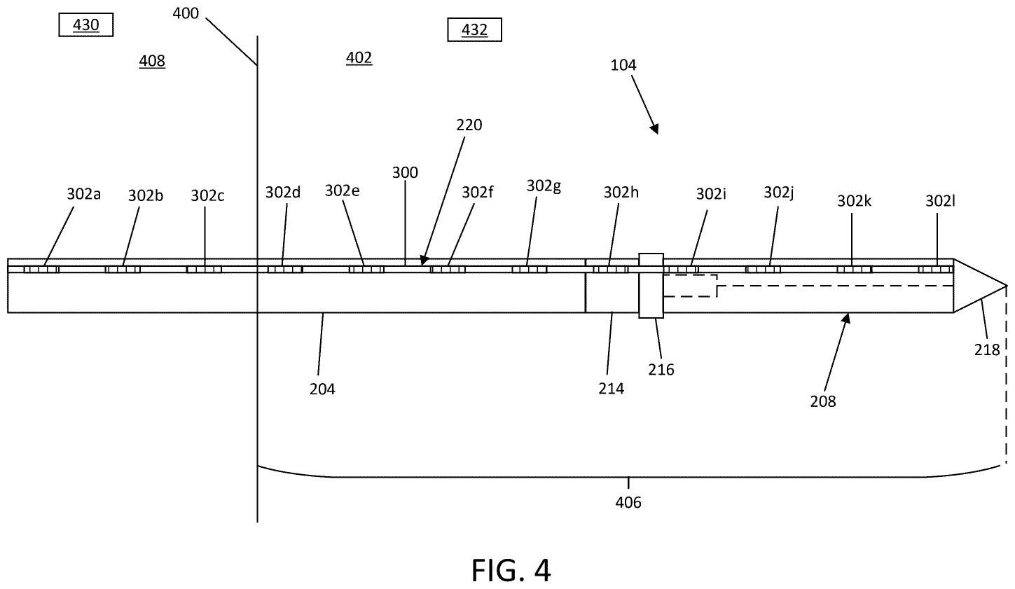

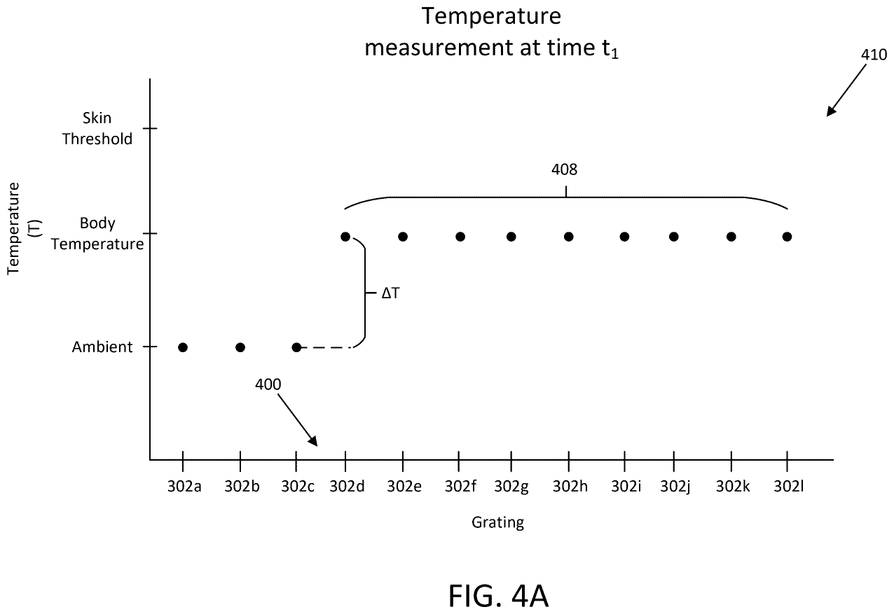

FIG. 4 is a schematic diagram showing the ablation probe 104 of FIG. 3 inserted through the skin 400 of a patient 402 at a first time t₁, according to at least one aspect of the present disclosure. As shown in FIG. 4, the cannula 204 and the antenna 208 of ablation probe 104 are inserted through the skin 400 to an insertion depth 406 such that a portion of the FBGs 302a-c remain exposed to an ambient environment 408 (e.g., the operating room) while the remaining FBGs 302d-l are positioned within the patient 402 beneath the skin 400.

In such a position, a user may desire to know the insertion depth 406 of the ablation probe 104 into the patient 402 to determine how close the antenna 208 is to the skin 400 and/or if the antenna 208 has reached a desired insertion depth prior to energizing the probe 104, for example. Accordingly, in some embodiments, the user can provide an input to an input interface, such as the GUI 120. Based on the input, the controller 114 can cause the optical interrogator 230 to transmit an optical signal along the optical fiber 300 and receive reflected signals therefrom. Based on the reflected signals, the optical interrogator 230 can determine the temperature at each of the FBGs 302a-l. In other embodiments, the controller 114 can automatically control the optical interrogator 230 to transmit the optical signal through the optical fiber 300, such as periodically or continuously.

FIG. 4A is a graph 410 of temperature measurements at each of the FBGs 302a-l at the first time t₁. Based on the measured temperatures of the FBGs 302a-l, the controller 114 can determine which of the FBGs 302a-l are exposed to the ambient environment 408 and which are inserted into the patient 402 to therefore determine the insertion depth 406 of the antenna 208.

In some embodiments, the system 100 further includes a reference temperature sensor usable to determine which FBGs are inserted into the patient 402 and which are exposed to the ambient environment 408. In some embodiments, the reference temperature sensor comprises an ambient temperature sensor 430 to measure a temperature of the ambient environment 408. In some such embodiments, the controller 114 can compare some, or all, of the measured temperatures of the FBGs 302a-l to the measured ambient temperature to determine which of the FBGs 302a-l are exposed to the ambient environment 408. For instance, in one embodiment, the controller 114 can sequentially compare the measured ambient temperature to the measured temperatures of the FBGs 302a-l, starting with the first FBG 302a temperature measurement and working toward the last FBG 302l temperature measurement. Once the controller 114 detects a threshold temperature difference ΔT between the measured ambient temperature and a measured temperature at an FBG, the controller 114 can cease comparing temperatures and determine that the FBG outputting the threshold temperature ΔT greater than the measured ambient temperature (as well as all distally positioned FBGs) are inserted within the patient 402. In another embodiment, the controller 114 compares each of the measured temperatures of the FBGs 302a-l to the measured ambient temperature to determine which of the FBGs 302a-l are the threshold temperature ΔT greater than the measured ambient temperature and, thus, which FBGs are inserted into the patient 402. In some embodiments, the threshold temperature ΔT is stored in the memory 117 and is retrievable by the controller 114.

In another embodiment, the controller 114 compares each of the measured temperatures of the FBGs 302a-l to the measured ambient temperature to determine which of the FBGs 302a-l are at, or within a threshold from, the measured ambient temperature to determine which FBGs are exposed to the ambient environment 408. Alternatively, or in combination with above, the reference temperature sensor comprises a body temperature sensor 432, such as a thermometer, for example, which can measure the body temperature of the patient 402. The controller 114 can compare each of the measured temperatures of the FBGs 302a-l to the measured body temperature to determine which of the FBGs 302a-l are at, or within a threshold from, the measured body temperature to determine which FBGs are inserted into the patient 402.

In some instances, the temperature of the ambient environment 408 may be the same, or at least substantially the same, as the tissue in which the probe 104 is inserted. As a result, the controller 114, using the temperature measurements from the FBGs 302a-l, may not be able to discern which of the FBGs are inserted into the patient 402 and which of the FBGs are exposed to the ambient environment 408. Accordingly, the controller 114 may provide coolant through the cooling tube 212 from the coolant source 107, such as a pulse or step function of coolant. As the coolant source 107 provides coolant to the cooling tube 212, the controller 114 may monitor the temperature of each of the FBGs 302a-l. In particular, the local rate of temperature change at each of the FBGs 302a-l in response to the coolant being provided may be monitored, where FBGs exposed to the ambient environment 408 will change temperature faster than FBGs contacting tissue, which acts as a heat sink. Accordingly, the controller 114 may determine which of the FBGs are inserted into the patient 402 and which of the FBGs are exposed to the ambient environment 408 based on a local rate of temperature change as coolant is provided to the probe 104 from the coolant source 107.

In other embodiments, the controller 114 may compare the measured temperature of each FBG to adjacent FBG measured temperatures to determine if there is a threshold temperature difference ΔT therebetween. For example, in one embodiment, the controller 114 can compare the measured temperature at FBG 302d to the measured temperature at proximally-positioned FBG 302c and distally-positioned 302e. Based on detecting FBG 302c measuring a temperature that is a threshold temperature difference ΔT less than the measured temperature at 302d, the controller 114 can determine that FBGs 302a-c are exposed to the ambient environment 408 and the remaining FBGs 302d-l are inserted into the patient 402. Accordingly, utilizing the FBGs 302a-l enables the controller 114 to ascertain where the skin 400 is located along the ablation probe 104.

Based on a determination of which FBGs are inserted into the patient 402, the controller 114 can determine the insertion depth 406 of the energy delivery device 104 into the patient 402. For instance, in some embodiments, the controller 114 can calculate the insertion depth 406 using the known lengths l_{FBG} of the FBGs, the known distances d_{FBD} between the FBGs, and the known length of the stylet 218. In some embodiments, the lengths l_{FBG}, the distances d_{FBD}, and the length of the stylet 218 are stored in the memory 117 and are retrievable by the controller 114. Referring to FIGS. 4 and 4A, based on the controller 114 detecting that FBGs 302d-l are inserted into the patient 402, the control system can retrieve the length l_{FBG} of FBGs 302d-l, the distances d_{FBD} between 302d,e, 302e,f . . . 302k,l, and the length of the stylet 218 and calculate the insertion depth 406 by adding all of these lengths and distances together. In some other embodiments, the memory 117 stores a look-up table which correlates detected inserted FBGs to an insertion depth 406 of the probe. For instance, in some such embodiments, the controller 114 can determine the insertion depth 406 by looking up, in the look-up table, the distance associated with having FBGs 302d-l inserted into the patient 402. In some embodiments, the controller 114 can display the insertion depth 406 on a display, such as the GUI 120, for the user to interpret.

In some embodiments, the controller 114 can determine the depth of each specific FBG into the patient 402 using the known lengths l_{FBG} of the FBGs and the known distances d_{FBD} between the FBGs. For instance, as referenced above, some of the FBGs may be strategically defined along the optical fiber 300 at key locations, such as at the stick region 214 (FBG 302h), at a proximal end 304 of the antenna 208 (FBG 302i), and a distal end 306 of the antenna 208 (302l). Based on the known lengths l_{FBG} of the FBGs and the known distances d_{FBD} between the FBGs, the controller 114 can further calculate the depth of the stick region 214 and the proximal/distal ends of the antenna 208 into the patient 402 and display these distances on the display (GUI 120). In some other embodiments, similar to the above, the memory 117 stores a look-up table which correlates detected inserted FBGs to depths of each FBG into the patient 402. For instance, in some such embodiments, the controller 114 can determine the insertion depth of each FBG, such as FBGs 302h,i,l, by looking up, in the look-up table, the distances associated with having FBGs 302d-l inserted into the patient 402.

In some embodiments, the controller 114 can prevent activation of the ablation probe 104 unless certain portions of the ablation probe 104 (stick region 214, proximal end of antenna 208, etc.) are positioned a threshold distance away from the skin 400. In some such embodiments, the threshold distances are stored in the memory 117 and retrievable by the controller 114. In some embodiments, the controller 114 can dynamically determine the insertion depth 406 by monitoring changes in the temperatures measured by the FBGs. For instance, a user may insert the ablation probe 104 further into the patient 402, causing the FBG 302c to move into the patient 402. The controller 114 can detect an increase in measured temperature at the FBG 302c and determine that the FBG 302c is now inserted into the patient 402. The controller 114 can thus dynamically update the measured insertion depth 406 according to the number of FBGs inserted into the patient 402, as detected by the controller 114.

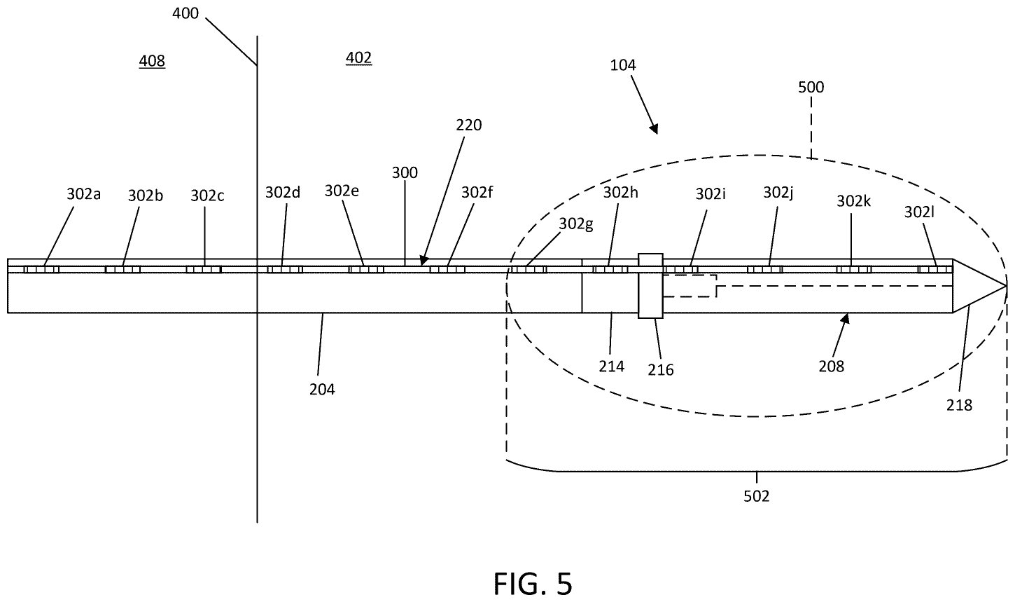

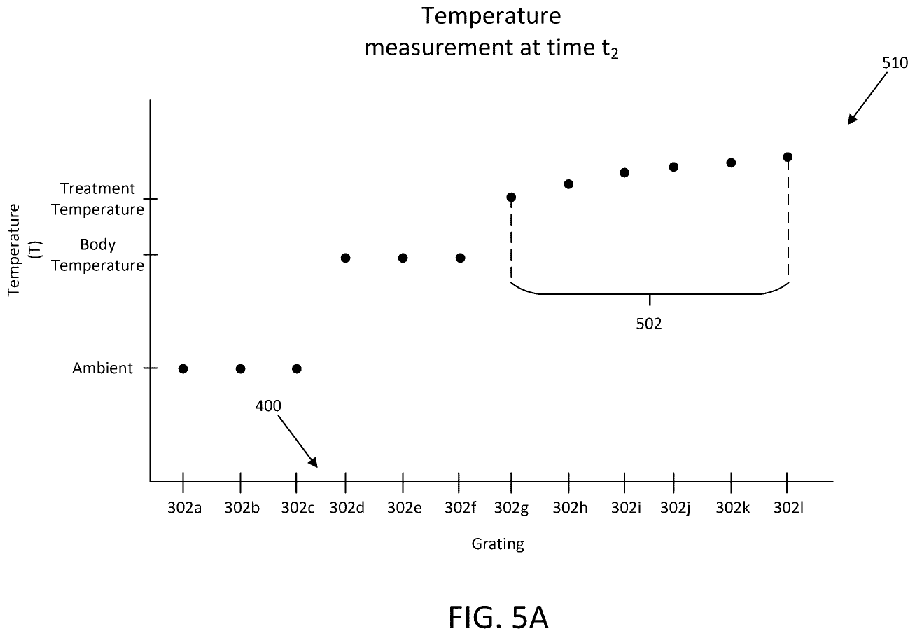

Once the user is satisfied with the position of the ablation probe 104 in the patient 402, the user may choose to activate the ablation probe 104 to emit energy from the antenna 208. FIG. 5 is the energy delivery device 104 of FIG. 4 inserted through the skin 400 of the patient 402 at a second time t₂ later than the first time t₁, according to at least one aspect of the present disclosure. As shown in FIG. 5, at t₂, the antenna 208 has been activated to emit energy to generate an ablation zone 500 with an ablation length 502 that encompasses FBGs 302g-l.

FIG. 5A is a graph 510 of temperature measurements at each of the FBGs 302a-l at the second time t₂. Based on the measured temperatures of the FBGs 302a-l, the controller 114 can determine and/or monitor the ablation length 502 of the generated ablation zone 502 along the ablation probe 104 over time. For instance, in some embodiments, the controller 114 can monitor the measured temperatures at each of the FBGs inserted into the patient 402. Based on the controller 114 detecting an increase in temperature at a particular FBG above a treatment temperature threshold, the controller 114 can determine that the generated ablation zone 500 has ablated the tissue adjacent the particular FBG. Accordingly, the controller 114 can dynamically calculate the ablation length 502 of the generated ablation zone 500 based on the detected increases in temperatures at the FBGs above the treatment temperature threshold. In some embodiments, the treatment temperature threshold is 60° C.

For instance, referring to graph 510, the controller 114 detects an increase in temperature at FBGs 302g-l above the treatment temperature threshold. Based on the detection, the controller 114 may calculate the ablation length 502 using the known lengths associated with the ablation probe 104, such as the known length l_{FBG} of the FBGs, the known distances d_{FBD} between the FBGs, and the known length of the stylet 218. In some embodiments, the lengths l_{FBG}, the distances d_{FBD}, and the length of the stylet 218 are stored in the memory 117 and are retrievable by the controller 114. Based on the controller 114 detecting that FBGs 302g-l increase in temperature above the treatment temperature threshold, the controller 114 can retrieve the length l_{FBG} of FBGs 302g-l, the length of the stylet 218, and the distances d_{FBD} between 302g,h, 302h,i . . . 302k,l and calculate the ablation length 502 by adding all of the distances d_{FBD}, lengths l_{FBG}, and stylet 218 length together. In some embodiments, the controller 114 can display the ablation length 502 on a display, such as the GUI 120, for the user to interpret.

In some embodiments, in addition to monitoring the ablation length 502, the controller 114 can monitor the insertion depth 406 (FIG. 4) and perform an action based on the ablation length 502 approaching, reaching, or exceeding the insertion depth 406. For instance, the ablation length 502 approaching the insertion depth 406 can indicate to a user and/or the controller 114 that the temperature of the skin 400 may soon increase, which may cause the skin 400 to burn. In some embodiments, the action comprises generating an alert, such as actuating an audio module (e.g., a speaker), actuating a tactile module (e.g., a motor in the handle 202), or displaying a warning on a display, such as the GUI 120. Alternatively, or in combination with the above, the action comprises increasing conveyance of cooling fluid to the stick region 214 and/or ceasing to provide energy to the antenna 208, as examples.

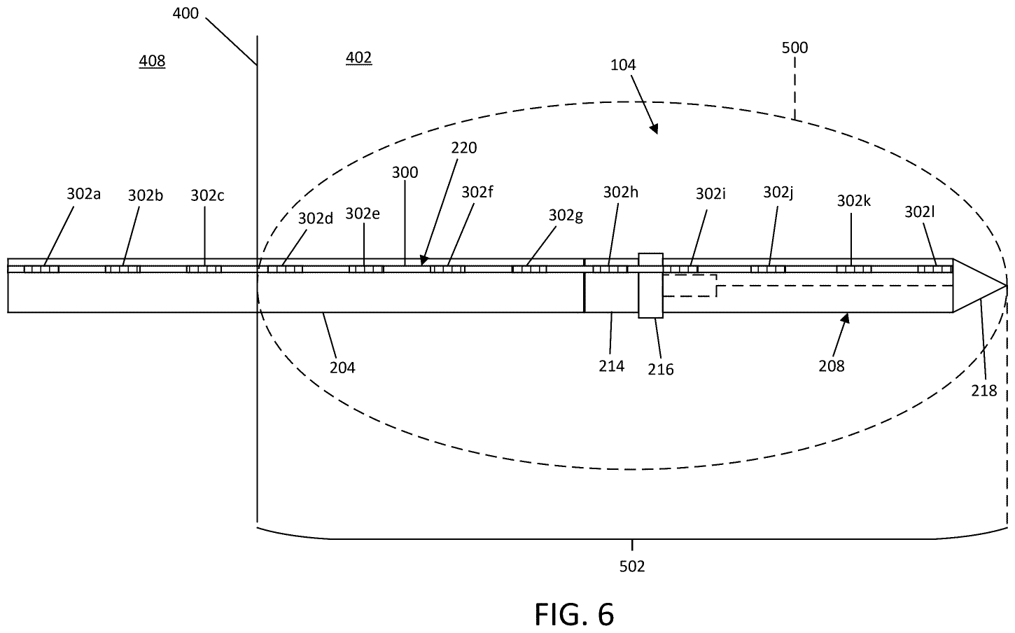

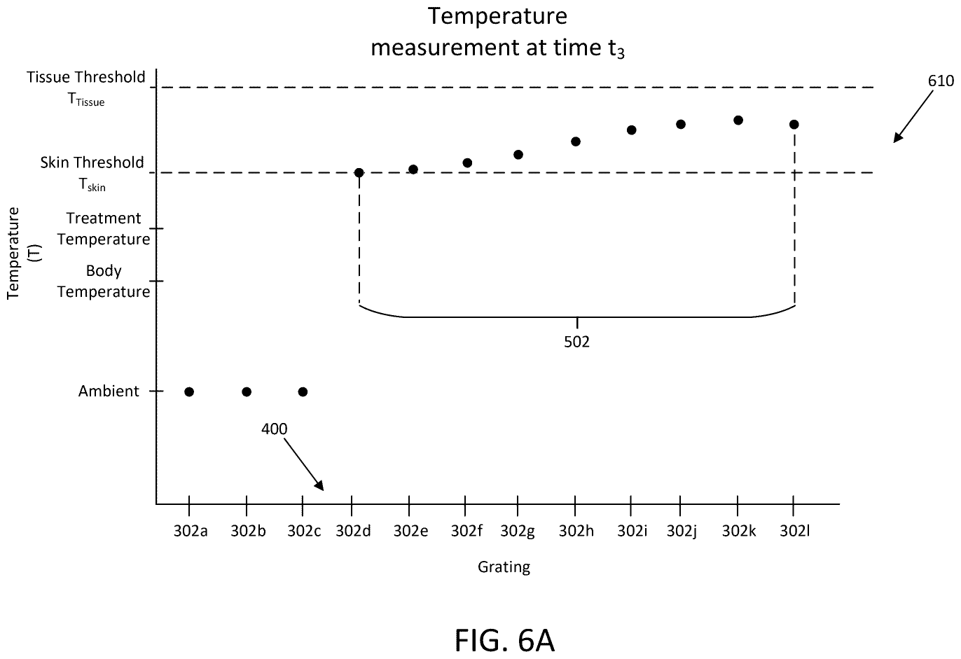

FIG. 6 is the ablation probe 104 of FIG. 4 inserted through the skin 400 of the patient 402 at a third time t₃ later than the second time t₂, according to at least one aspect of the present disclosure. As shown in FIG. 6, at t₃, the generated ablation zone 500 and, thus, the ablation length 502, has grown and now encompasses FBGs 302e-l and proximal-most inserted FBG 302d.

In some embodiments, as discussed above, the controller 114 can determine which FBGs are exposed to the ambient environment 408 and which FBGs are inserted into the patient 402. Based on this determination, the controller 114 can determine which FBG is positioned within the patient 402 and closest to the skin 400 (i.e., the “proximal-most inserted FBG”). The controller 114 can utilize temperature measurement from the proximal-most inserted FBG as a proxy for the measured temperature of the skin 400. For instance, with respect to the embodiment of FIG. 6, the controller 114 can determine that FBGs 302d-l are positioned in the patient 402 and, thus, 302d is the proximal-most inserted FBG and can be used as a proxy for measuring the temperature of the skin 400. Utilizing this FBG allows the controller 114 to ensure that the skin 400 is not heated above a threshold skin temperature, which could result in the skin 400 burning.

FIG. 6A is a graph 610 of temperature measurements at each of the FBGs 302a-l at the third time t₃. Based on the measured temperatures of the FBGs 302a-l, the controller 114 can determine and/or monitor the temperature at each of the FBGs inserted into the patient 402. For instance, in some embodiments, the controller 114 can monitor the measured temperature at the proximal-most inserted FBG to ensure that the measured temperature does not reach or exceed a skin threshold temperature T_{Skin}. Based on the controller 114 detecting the measured temperature of the proximal-most inserted FBG approaching, reaching, or exceeding the skin threshold temperature T_{Skin}, the controller 114 can perform an action (actuate an alert, increase conveyance of coolant to stick region 214, or shut down ablation probe 104, or combinations thereof, as examples). In some embodiments, the skin temperature threshold T_{Skin} is stored in the memory 117 and is retrievable by the controller 114.

In some embodiments, the controller 114 can monitor the measured temperature at each of the inserted FBGs to ensure that the measured temperatures do not reach or exceed a tissue threshold temperature T_{Tissue}. Based on the controller 114 detecting at least one of the inserted FBGs approaching, reaching, or exceeding the tissue threshold temperature T_{Tissue}, the controller 114 can perform an action (actuate an alert, increase conveyance of coolant to stick region 214, or shut down ablation probe 104, or combinations thereof, as examples). In some embodiments, the tissue temperature threshold T_{Tissue} is stored in the memory 117 and is retrievable by the controller 114.

As shown in graph 610, at t₃ the controller 114 detects the proximal-most inserted FBG 302 has reached the skin threshold temperature T_{Tissue}, which may result in the skin 400 burning. Accordingly, based on the detection, the controller 114 can perform an action to prevent the skin 400 from burning, such as actuating an alert, increasing conveyance of coolant to stick region 214, or shutting down ablation probe 104, or combinations thereof, as examples.

An ablation procedure with the ablation probe 104 typically includes two portions. The first portion, as described above, is an ablation period in which the ablation probe 104 generates an ablation zone to ablate tissue within a patient.

The second portion is a cauterization period or process in which energy is provided to the antenna 208 as the cannula 204 is manually withdrawn from the patient by a user, such as the clinician. The cauterization period is performed such that seed tumor cells are destroyed along the exit path of the cannula 204 as the ablation probe 104 is withdrawn from the patient. During this cauterization period, an important factor is the rate of withdrawal from the patient. In particular, the controller 114 may indicate to a user that, for an effective cauterization period, the ablation probe 104 should be withdrawn at a base withdrawal rate, such as 0.2 cm/sec, for example. Due to this withdrawal being performed by the user, the rate of withdrawal can vary during the cauterization period, such as withdrawing too fast or too slow with respect to the desired base withdrawal rate. Accordingly, it is desirable to provide a system that provides the user with feedback regarding the rate of withdrawal during the cauterization period.

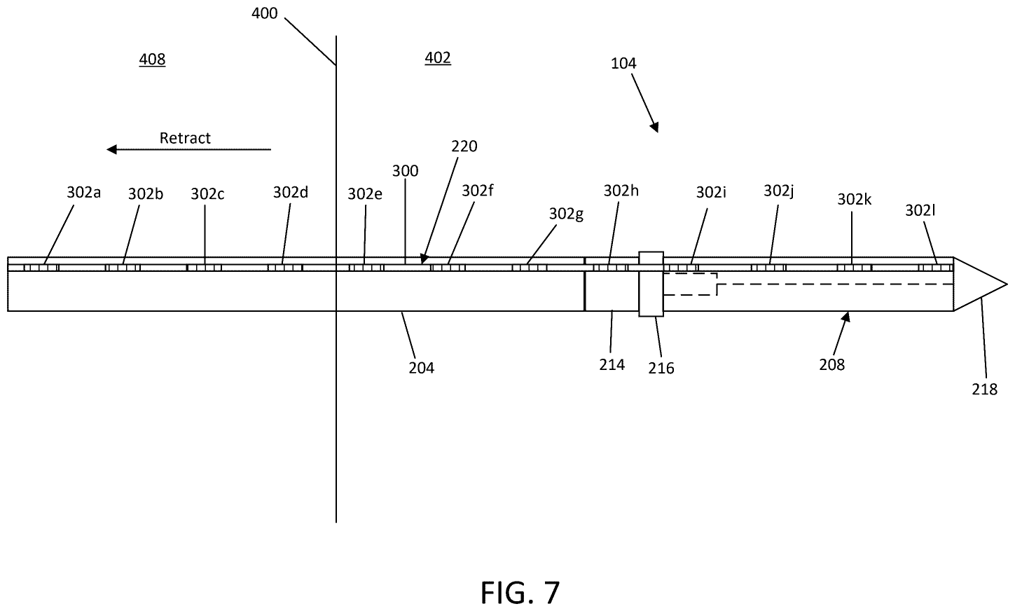

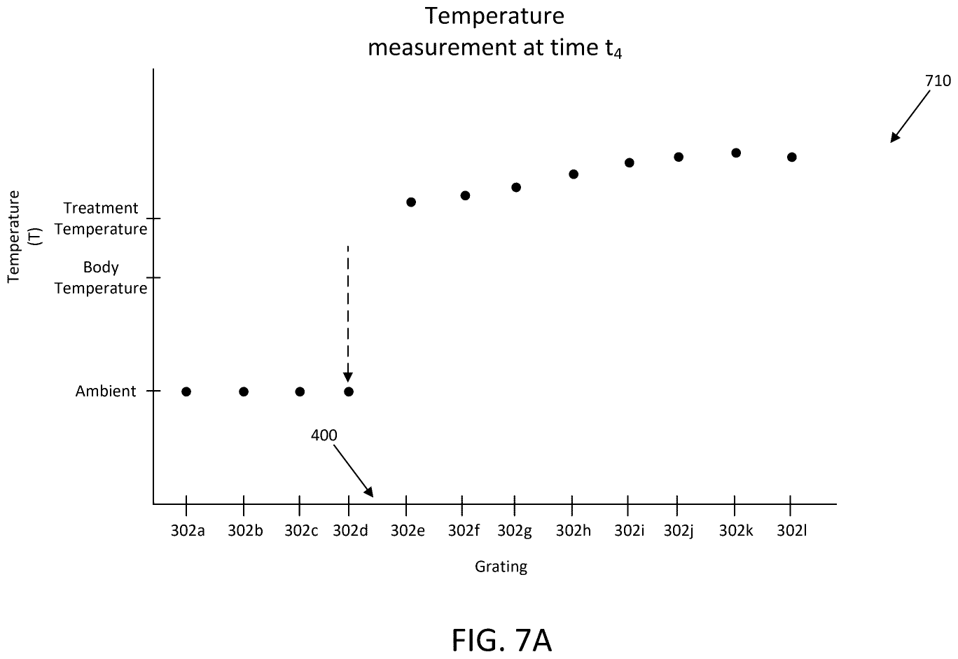

FIG. 7 is the ablation probe 104 of FIG. 4 inserted through the skin 400 of the patient 402 at a fourth time t₄ later than the third time t₃, according to at least one aspect of the present disclosure. As shown in FIG. 7, at t₄, the ablation probe 104 has been retracted such that the proximal-most inserted FBG 302d has been withdrawn through the skin 400 and is now exposed to the ambient environment 408 (making FBG 302e the new proximal-most inserted FBG).

FIG. 7A is a graph 710 of temperature measurements at each of the FBGs 302a-l at the fourth time t₄. Based on the measured temperatures of the FBGs 302a-l, the controller 114 can determine and/or monitor the rate of movement (velocity) of the ablation probe 104 with respect to (through) the skin 400, such as the rate of probe withdrawal during the cauterization period. In particular, as shown in FIG. 7A, as the proximal-most inserted FBG 302c is withdrawn from the patient 402, the temperature measured by the FBG 302c sharply drops to ambient temperature (as measured by the ambient temperature sensor 430 (FIG. 4), for example). The controller 114 can detect this drop in temperature and determine that the ablation probe 104 is being withdrawn from the patient 402.

In some embodiments, based on the detection of the ablation probe 104 being withdrawn from the patient 402, the controller 114 can calculate the rate of movement using the known lengths associated with the ablation probe 104, such as the known length l_{FBG} of the FBGs, the known distances d_{FBD} between the FBGs, for example. For instance, referring to FIG. 7A, in some embodiments, the controller 114 can detect the sharp temperature drop at FBG 302c to ambient and estimate the rate of withdrawal as being (l_{FBG}+d_{FBG})/(t₄−t₃). In some embodiments, the controller 114 can display the rate of movement on a display, such as the GUI 120, for the user to monitor as they withdrawal the ablation probe 104 from the patient 402.

In some embodiments, the controller 114 can compare the detected rate of withdrawal to the desired base withdrawal rate. Based on the rate of withdrawal being a threshold distance from the desired base withdrawal rate (i.e., detecting that the ablation probe 104 moving too fast or too slow), the control system 102 can perform an action, such as actuating an alert (audio, tactile, or visual, for example) telling the user to speed up or slow down, or shutting down the ablation probe 104. Accordingly, the optical sensor 220 enables a user to dynamically track the rate of probe removal during the cauterization period.

Furthermore, as discussed above, the controller 114 can monitor the position of each FBG, such as the strategically defined FBG at the stick region 214 (FBG 302h), the FBG at a proximal end 304 of the antenna 208 (FBG 302i), and the FBG at the distal end 306 of the antenna 208 (302l), as examples. In some embodiments, the controller 114 can monitor the position of the FBGs during the cauterization period and perform an action according to their determined position.

For instance, the stick region 214 approaching, reaching, or exceeding a threshold distance from the skin 400 may cause the skin 400 to freeze, thus damaging the skin 400. Accordingly, during the cauterization period, the controller 114 can monitor a position of the FBG associated with the stick region (FBG 302h) and cease operating of the ablation probe 104 based on the FBG 302h approach, reaching, or exceeding the threshold distance from the skin 400. Alternatively, in some embodiments, the controller 114 can cease providing coolant to the stick region 214 based on the FBG 302h approach, reaching, or exceeding the threshold distance from the skin 400, but continue to provide energy to the antenna 208. In some embodiments, the threshold distance is stored in the memory 117 and is retrievable by the controller 114.

For another instance, the proximal end of the antenna 208 approaching, reaching, or exceeding a threshold distance from the skin 400 may cause the skin 400 to burn, thus damaging the skin 400. Accordingly, in some embodiments, or in combination with those discussed above, during the cauterization period, the controller 114 can monitor a position of the FBG associated with the proximal end of the antenna 208 (FBG 302i) and cease operating of the ablation probe 104 based on the FBG 302i approach, reaching, or exceeding the threshold distance from the skin 400. In some embodiments, the threshold distance is stored in the memory 117 and is retrievable by the controller 114.

The controller 114 may monitor intentional movement of the ablation probe 104, such as movement of the ablation probe 104 during the cauterization period, as discussed elsewhere herein. The controller 114 may also monitor unintentional movement of the ablation probe 104. As one example, during ablation, the stick region 214 may be “turned off” (i.e., coolant delivery to the stick region 214 ceases) and the ablation probe 104 may inadvertently be moved prior to the tissue around the probe 104 charring enough to lock the probe 104 in place. As another example, the ablation probe 104 may be inadvertently bumped by a clinician during the ablation procedure, causing the ablation probe 104 to move from its intended location. Accordingly, the controller 114 may monitor movement of the ablation probe 104 while the ablation probe 104 is providing energy to the tissue.

For instance, referring again to FIG. 2-4, the controller 114 may determine the insertion depth 406 of the antenna 208 using the FBGs 302a-l, as discussed elsewhere herein. The controller 114 may monitor the insertion depth 406 and detect changes thereof based on the antenna 208 providing energy to the tissue. Based on a change in the insertion depth 406 approaching, reaching, or exceeding a depth change threshold, the controller 114 may perform an action. The action may comprise generating an alert, such as actuating an audio module (e.g., a speaker), actuating a tactile module (e.g., a motor in the handle 202), or displaying a warning on a display, such as the GUI 120, or combinations thereof. Alternatively, or in combination with the above, the action may comprise increasing conveyance of cooling fluid to the stick region 214 and/or ceasing to provide energy to the antenna 208. The depth change threshold may be stored in the memory 117 and may be retrievable by the controller 114.

Microwave ablation has become increasingly popular to address pain management for bone metastasis. However, there have been complaints surrounding mechanical breaks on the stylet, like stylet 218, and/or the cannula, like cannula 204. Since the user is inserting the probe, like probe 104, into bone, as opposed to soft tissue, there is a chance that the user may break the probe during placement.

As discussed elsewhere herein, the controller 114 may monitor the reflected wavelengths from the FBGs 302a-l to determine the strain experienced by each of the FBGs 302a-l. The controller 114 may monitor the determined strain at each of the FBGs 302a-l and detect changes thereof, such as when the probe 104 is being inserted into a patient or when the antenna 208 is providing energy to the tissue, for example. Based on a change in one or more of the determined strains approaching, reaching, or exceeding a strain threshold, the controller 114 may perform an action. The action may comprise generating an alert, such as actuating an audio module (e.g., a speaker), actuating a tactile module (e.g., a motor in the handle 202), or displaying a warning on a display, such as the GUI 120, or combinations thereof. The warning on the display may include a message indicating to the user to not bend the probe 104. Alternatively, or in combination with the above, the action may comprise ceasing to provide energy to the antenna 208. The strain threshold may be stored in the memory 117 and may be retrievable by the controller 114.

As discussed elsewhere herein, the controller 114 may monitor the local rate of temperature change at each of the FBGs 302a-l. This may be particularly beneficial for monitoring the ablation zone at the end of an ablation treatment. More specifically, the immediate temperature at each of the FBGs at the end of the ablation treatment is a mix of tissue and coolant effects. The controller 114 may monitor the temperature rate of decay at each of the FBGs 302a-l after ceasing flow of coolant to determine the amount of hot tissue surrounding the probe. For instance, an FBG embedded in a first amount of hot tissue (e.g., several centimeters) will have a first rate of temperature decay, whereas an FBG embedded in a second amount of hot tissue less than the first amount of hot tissue (e.g. a millimeter) will have a second rate of temperature decay greater than the first amount of how tissue.

Embodiments disclosed herein include:

A. A system comprising an energy delivery device and a controller. The energy delivery device includes a cannula, an antenna extending from the cannula and operable to deliver energy to tissue of a patient, the cannula and the antenna being insertable through skin of the patient to an insertion depth, and an optical sensor including Fiber Bragg Gratings (FBGs). The controller is in communication with the optical sensor and operable to receive temperature measurements from the optical sensor and determine the insertion depth based on the received temperature measurements.

B. A method comprising inserting an antenna and a cannula of an energy delivery device through the skin of a patient to an insertion depth, transmitting an optical signal through an optical sensor of the energy delivery device, receiving a plurality of reflected optical signals from the optical sensor reflected off Fiber Bragg Gratings (FBGs) defined on the optical sensor, determine a temperature at each of the FBGs based on the reflected optical signals, and determining the insertion depth of the energy delivery device based on the determined temperatures.

C. A system comprising an energy delivery device and a controller. The energy delivery device includes a cannula insertable through skin of a patient, an antenna extending from the cannula and operable to operable to deliver energy to tissue of the patient, and an optical sensor. The controller is in communication with the optical sensor and operable to receive temperature measurements from the temperature sensor and determine a rate of movement of the energy delivery device relative to the skin based on the received temperature measurements.

Each of embodiments A, B, and C may have one or more of the following additional elements in any combination: Element 1: wherein the controller is further operable to detect a change in the insertion depth that exceeds a depth change threshold as the antenna delivers energy to the tissue and perform an action based on the detection. Element 2: wherein the action comprises ceasing energy delivery to the energy delivery device. Element 3: wherein the antenna is operable to generate an ablation zone based on the antenna delivering energy to the tissue, and the controller is operable to determine a length of the ablation zone along the energy delivery device based on the received temperature measurements. Element 4: wherein the controller is further operable to compare the determined length of the ablation zone to the insertion depth and perform an action based on the comparison. Element 5: wherein the action comprises ceasing energy delivery to the antenna. Element 6: wherein the controller is further operable to receive a strain measurement from the optical sensor, detect a change in the strain measurement that exceeds a strain threshold, and perform an action based on the detection. Element 7: wherein the action comprises generating an alert on a display. Element 8: wherein the optical sensor comprises a first temperature sensor portion to measure a first temperature and a second temperature sensor portion to measure a second temperature, wherein the controller is further operable to detect a threshold temperature difference between the first and second measured temperatures, and wherein determining the insertion depth is based on detecting the threshold temperature difference between the first and second temperature measurements. Element 9: wherein the system further comprises a reference temperature sensor to measure a reference temperature, and the optical sensor comprises a first temperature sensor portion to sense a first temperature and a second temperature sensor portion to sense a second temperature, wherein the controller is further to compare the first and second temperature measurements to the measured reference temperature, and wherein determining the insertion depth is based on the comparison. Element 10: wherein the reference temperature sensor comprises an ambient temperature sensor. Element 11: wherein the reference temperature sensor comprises a body temperature sensor. Element 12: further comprising energizing the energy delivery device to generate an ablation zone at the antenna and determining a length of the ablation zone along the energy delivery device based on the determined temperatures. Element 13: further comprising comparing the determined length of the ablation zone to the insertion depth and performing an action based on the comparison. Element 14: further comprising determining a strain at each of the FBGs based on the reflected optical signals, detecting a change in the strain that exceeds a strain threshold, and perform an action based on the detection. Element 15: wherein the system further comprises a reference temperature sensor to measure a reference temperature, wherein the optical sensor comprises a temperature sensor portion, and wherein the controller is further operable to detect, at a first time with the temperature sensor portion, a first temperature greater than the measured reference temperature and detect, at a second time with the temperature sensor portion, the first temperature dropping toward the measured reference temperature, wherein determining the rate of movement is based on detecting the first temperature dropping toward the measured reference temperature at the second time. Element 16: wherein the controller is further operable to receive a strain measurement from the optical sensor, detect a change in the strain measurement that exceeds a strain threshold, and perform an action based on the detection. Element 17: wherein the action comprises generating an alert on a display.

By way of non-limiting example, exemplary combinations applicable to A, B, and C include: Element 1 with Element 2; Element 1 with Element 3; Elements 1 and 2 with Element 3; Element 1 with Elements 3 and 4; Elements 1 and 2 with Elements 3 and 4; Element 1 with Elements 3-5; Elements 1 and 2 with Elements 3-5; Element 1 with Element 6; Elements 1 and 2 with Element 6; Element 1 with Elements 6 and 7; Elements 1 and 2 with Elements 6 and 7; Element 1 with Element 8; Elements 1 and 2 with Element 8; Element 1 with Element 9; Elements 1 and 2 with Element 9; Element 1 with Elements 9 and 10; Elements 1 and 2 with Elements 9 and 10; Element 1 with Elements 9 and 11; Elements 1 and 2 with Elements 9 and 11; Element 3 with Element 4; Element 3 with Elements 4 and 5; Element 3 with Element 6; Elements 3 and 4 with Element 6; Elements 3-5 with Element 6; Element 3 with Elements 6 and 7; Elements 3 and 4 with Elements 6 and 7; Elements 3-5 with Elements 6 and 7; Element 3 with Element 8; Elements 3 and 4 with Element 8; Elements 3-5 with Element 8; Element 3 with Element 9; Elements 3 and 4 with Element 9; Elements 3-5 with Element 9; Element 3 with Elements 9 and 10; Elements 3 and 4 with Elements 9 and 10; Elements 3-5 with Elements 9 and 10; Element 3 with Elements 9 and 11; Elements 3 and 4 with Elements 9 and 11; Elements 3-5 with Elements 9 and 11; Element 6 with Element 7; Element 6 with Element 8; Elements 6 and 7 with Element 8; Element 6 with Element 9; Elements 6 and 7 with Element 9; Element 6 with Elements 9 and 10; Elements 6 and 7 with Elements 9 and 10; Element 6 with Elements 9 and 11; Elements 6 and 7 with Elements 9 and 11; Element 8 with Element 9; Element 8 with Elements 9 and 10; Element 8 with Elements 9 and 11; Element 9 with Element 10; Element 9 with Element 11; Element 12 with Element 13; Element 12 with Elements 13 and 14; Element 15 with Element 16; Element 15 with Elements 16 and 17; and Element 16 with Element 17.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. A system, comprising:
an energy delivery device that includes:
a cannula;
an antenna extending from the cannula and operable to deliver energy to tissue of a patient, the cannula and the antenna being insertable through skin of the patient to an insertion depth; and
an optical sensor including Fiber Bragg Gratings (FBGs); and
a controller in communication with the optical sensor and operable to:
receive temperature measurements from the optical sensor; and
determine the insertion depth based on the received temperature measurements.

2. The system of claim 1, wherein the controller is further operable to:
detect a change in the insertion depth that exceeds a depth change threshold as the antenna delivers energy to the tissue; and
perform an action based on the detection.

3. The system of claim 2, wherein the action comprises ceasing energy delivery to the energy delivery device.

4. The system of claim 1, wherein the antenna is operable to generate an ablation zone based on the antenna delivering energy to the tissue, and the controller is operable to determine a length of the ablation zone along the energy delivery device based on the received temperature measurements.

5. The system of claim 4, wherein the controller is further operable to:
compare the determined length of the ablation zone to the insertion depth; and
perform an action based on the comparison.

6. The system of claim 5, wherein the action comprises ceasing energy delivery to the antenna.

7. The system of claim 1, wherein the controller is further operable to:
receive a strain measurement from the optical sensor;
detect a change in the strain measurement that exceeds a strain threshold; and
perform an action based on the detection.

8. The system of claim 7, wherein the action comprises generating an alert on a display.

9. The system of claim 1, wherein the optical sensor comprises:
a first temperature sensor portion to measure a first temperature; and
a second temperature sensor portion to measure a second temperature,
wherein the controller is further operable to detect a threshold temperature difference between the first and second measured temperatures, and
wherein determining the insertion depth is based on detecting the threshold temperature difference between the first and second temperature measurements.

10. The system of claim 1, wherein the system further comprises a reference temperature sensor to measure a reference temperature, and the optical sensor comprises:
a first temperature sensor portion to sense a first temperature; and
a second temperature sensor portion to sense a second temperature,
wherein the controller is further to compare the first and second temperature measurements to the measured reference temperature, and
wherein determining the insertion depth is based on the comparison.

11. The system of claim 10, wherein the reference temperature sensor comprises an ambient temperature sensor.

12. The system of claim 10, wherein the reference temperature sensor comprises a body temperature sensor.

13. A method, comprising:
inserting an antenna and a cannula of an energy delivery device through the skin of a patient to an insertion depth;
transmitting an optical signal through an optical sensor of the energy delivery device;
receiving a plurality of reflected optical signals from the optical sensor reflected off Fiber Bragg Gratings (FBGs) defined on the optical sensor;
determine a temperature at each of the FBGs based on the reflected optical signals; and
determining the insertion depth of the energy delivery device based on the determined temperatures.

14. The method of claim 13, further comprising:
energizing the energy delivery device to generate an ablation zone at the antenna; and
determining a length of the ablation zone along the energy delivery device based on the determined temperatures.

15. The method of claim 14, further comprising:
comparing the determined length of the ablation zone to the insertion depth; and
performing an action based on the comparison.

16. The method of claim 13, further comprising:
determining a strain at each of the FBGs based on the reflected optical signals;
detecting a change in the strain that exceeds a strain threshold; and
perform an action based on the detection.

17. A system, comprising:
an energy delivery device that includes:
a cannula insertable through skin of a patient;
an antenna extending from the cannula and operable to operable to deliver energy to tissue of the patient; and
an optical sensor; and
a controller in communication with the optical sensor and operable to:
receive temperature measurements from the optical sensor; and
determine a rate of movement of the energy delivery device relative to the skin based on the received temperature measurements.

18. The system of claim 17, wherein the system further comprises a reference temperature sensor to measure a reference temperature, wherein the optical sensor comprises a temperature sensor portion, and wherein the controller is further operable to:
detect, at a first time with the temperature sensor portion, a first temperature greater than the measured reference temperature; and
detect, at a second time with the temperature sensor portion, the first temperature dropping toward the measured reference temperature,
wherein determining the rate of movement is based on detecting the first temperature dropping toward the measured reference temperature at the second time.

19. The system of claim 17, wherein the controller is further operable to:
receive a strain measurement from the optical sensor;
detect a change in the strain measurement that exceeds a strain threshold; and
perform an action based on the detection.

20. The system of claim 19, wherein the action comprises generating an alert on a display.
